(19) United States
(12) Patent Application Publication
(10) Pub. No.: US 2021/0247381 A1
(43) Pub. Date: Aug. 12, 2021

(54) APPARATUS AND METHOD FOR ANALYZING EXHALED BREATH

(71) Applicant: SAMSUNG ELECTRONICS CO., LTD., Suwon-si (KR)

(72) Inventors: Won Jong Jung, Seoul (KR); Kak Namkoong, Seoul (KR); Yeol Ho Lee, Anyang-si (KR); Joon Hyung Lee, Seongnam-si (KR); Ki Young Chang, Seoul (KR)

(73) Assignee: SAMSUNG ELECTRONICS CO., LTD., Suwon-si (KR)

(21) Appl. No.: 16/906,225

(22) Filed: Jun. 19, 2020

(30) Foreign Application Priority Data
Feb. 7, 2020 (KR) 10-2020-0014818

(51) Int. Cl. G01N 33/497; G01N 27/12

(52) U.S. Cl. CPC G01N 33/497; G01N 27/122; G01N 27/125

(57) ABSTRACT

An apparatus for analyzing an exhaled breath according to an example embodiment includes: a sensor configured to obtain at least one signal related to the exhaled breath, the at least one signal including a first signal indicating a target gas contained in the exhaled breath; and a processor configured to obtain a concentration of the target gas based on at least one signal feature value of the at least one signal and shape information over time of the at least one signal.

### CROSS-REFERENCE TO RELATED APPLICATION(S)

This application claims priority from Korean Patent Application No. 10-2020-0014818, filed on Feb. 7, 2020, in the Korean Intellectual Property Office, the entire disclosure of which is incorporated herein by reference for all purposes.

### BACKGROUND

### 1. Field

Example embodiments of the disclosure relate to an apparatus and a method for analyzing a target gas concentration in an exhaled breath.

### 2. Description of the Related Art

Gas sensors for measuring a concentration of a specific gas generally use a method of measuring a gas concentration based on a change in electric resistance caused by gas molecules adsorbed or desorbed to or from the surface of the gas sensors.

Particularly, in a normal environment of measuring gases contained in a person's exhaled breath, the accuracy of the gas sensors may be reduced by the surrounding environment such as humidity, temperature, and the like.

### SUMMARY

One or more example embodiments provide an apparatus and a method for analyzing an exhaled breath (e.g., analyzing a concentration of a target gas contained in the exhaled breath) with high accuracy by considering at least feature of a surrounding environment in which the exhaled breath is applied.

According to an aspect of an example embodiment, there is provided an apparatus for analyzing an exhaled breath, the apparatus including: a sensor configured to obtain at least one signal related to the exhaled breath, the at least one signal including a first signal indicating a target gas contained in the exhaled breath; and a processor configured to obtain a concentration of the target gas based on at least one signal feature value of the at least one signal and shape information over time of the at least one signal.

The at least one signal feature value may include at least one of an initial value of a feature obtained by the sensor immediately before the exhaled breath is applied, a maximum value of the feature obtained by the sensor from the exhaled breath, a minimum value of the feature obtained by the sensor from the exhaled breath, a difference value between the maximum value and the minimum value, a ratio between the initial value and the maximum value, a ratio between the initial value and the minimum value, and a ratio between the initial value and the difference value.

The shape information may include at least one of an area of a section in a waveform of the at least one signal between a first reference point and a point after a lapse of a first unit time from the first reference point, an average slope between a second reference point and a point after a lapse of a second unit time from the second reference point, and an instant slope at a third reference point.

The processor may be further configured to obtain a plurality of section areas, a plurality of average slopes, and a plurality of instant slopes, by moving the first reference point, the second reference point, and the third reference point in units of a predetermined period of time from an initial time of obtaining the at least one signal.

The processor may be further configured to obtain the concentration of the target gas by applying a pre-defined target gas estimation equation to the at least one signal feature value and the shape information.

The sensor may include a gas sensor, the gas sensor including a sensing layer, and wherein an electric resistance of the sensing layer changes by an oxidation reaction or reduction reaction between the sensing layer and a target gas molecule.

The sensing layer may include at least one of a metal oxide semiconductor (MOS), a graphene, a graphene oxide, a carbon nano tube (CNT), a conductive polymer, and a compound thereof.

The sensing layer may include a metal catalyst.

The sensing layer may include a nanostructure, the nanostructure including at least one of a nanofiber, a nanotube, a nanoparticle, a nanosphere, and a nanobelt.

The nanofiber may include a metal oxide semiconductor nanofiber, based on uniform binding of an alkali metal and a metal nanoparticle catalyst.

The gas sensor may include a signal electrode and a heater electrode, which are coated with the sensing layer.

The sensor may include at least one of a temperature sensor configured to obtain a temperature of the exhaled breath, a humidity sensor configured to obtain a humidity of the exhaled breath, and a pressure sensor configured to obtain a pressure of the exhaled breath.

The processor may be further configured to determine whether the exhaled breath is applied based on comparison of at least one of the temperature, the humidity, and the pressure of the exhaled breath with a predetermined threshold value.

The sensor may include the pressure sensor, and the processor is further configured to, based on the pressure of the exhaled breath obtained by the pressure sensor, determine at least one of whether the exhaled breath is normally applied, an initial time of obtaining the at least one signal, and a position at which the exhaled breath is applied.

The processor may be further configured to, based on upon a determination that the exhaled breath is not normally applied, provide information for guiding a user to re-apply the exhaled breath.

In accordance with an aspect of an example embodiment, there is provided a method of analyzing an exhaled breath, the method including: sensing, by a sensor, at least one signal related to the exhaled breath, the at least one signal including a first signal indicating a target gas contained in the exhaled breath; obtaining at least one signal feature value of the at least one signal and shape information over time of the at least one signal; and obtaining a concentration of the target gas based on the at least one signal feature value and the shape information.

The obtaining the concentration of the target gas may include obtaining the concentration of the target gas by applying a pre-defined target gas estimation equation to the at least one signal feature value and the shape information.

The method may further include determining whether the exhaled breath is applied, based on comparison of at least one of a temperature of the exhaled breath, a humidity of the exhaled breath, and a pressure of the exhaled breath with a predetermined threshold value.

The method may further include obtaining a pressure of the exhaled breath by using a pressure sensor, and determining, based on the pressure of the exhaled breath, at least one of whether the exhaled breath is normally applied, an initial time of obtaining the at least one signal, and a position at which the exhaled breath is applied.

The method may further include, in response to a determination that the exhaled breath is not normally applied, providing information for guiding a user to re-apply the exhaled breath.

### BRIEF DESCRIPTION OF THE DRAWINGS

These and/or other aspects will become apparent and more readily appreciated from the following description of the example embodiments, taken in conjunction with the accompanying drawings.

FIG. 1 is a block diagram illustrating an apparatus for analyzing an exhaled breath according to an example embodiment.

FIG. 2 is a block diagram illustrating a configuration of a sensor according to an example embodiment.

FIGS. 3A, 3B, and 3C are diagrams illustrating examples of a structure of a sensor according to example embodiments.

FIG. 4 is a diagram illustrating an example of an exhaled breath signal according to an example embodiment.

FIG. 5 is a block diagram illustrating an apparatus for analyzing an exhaled breath according to another example embodiment.

FIG. 6 is a flowchart illustrating a method of analyzing an exhaled breath according to an example embodiment.

FIG. 7 is a flowchart illustrating a method of analyzing an exhaled breath according to another example embodiment.

FIG. 8 is a flowchart illustrating a method of analyzing an exhaled breath according to yet another example embodiment.

FIGS. 9A, 9B, and 9C are diagrams illustrating a smart device according to an example embodiment.

### DETAILED DESCRIPTION

Details of example embodiments are included in the following detailed description and drawings. Advantages and features of the disclosure, and a method of achieving the same will be more clearly understood from the following example embodiments described in detail with reference to the accompanying drawings. Throughout the drawings and the detailed description, unless otherwise described, the same drawing reference numerals will be understood to refer to the same elements, features, and structures. The relative size and depiction of these elements may be exaggerated for clarity, illustration, and convenience.

It will be understood that, although the terms first, second, etc. may be used herein to describe various elements, these elements should not be limited by these terms. These terms are only used to distinguish one element from another. Any references to singular may include plural unless expressly stated otherwise. In addition, unless explicitly described to the contrary, an expression such as “comprising” or “including” will be understood to imply the inclusion of stated elements but not the exclusion of any other elements. Also, the terms, such as ‘part’ or ‘module’, etc., should be understood as a unit for performing at least one function or operation and that may be embodied as hardware, software, or a combination thereof.

As used herein, the term “and/or” includes any and all combinations of one or more of the associated listed items. Expressions such as “at least one of,” when preceding a list of elements, modify the entire list of elements and do not modify the individual elements of the list. For example, the expression, “at least one of a, b, and c,” should be understood as including only a, only b, only c, both a and b, both a and c, both b and c, or all of a, b, and c.

Hereinafter, example embodiments of an apparatus and method for analyzing an exhaled breath will be described in detail with reference to the accompanying drawings.

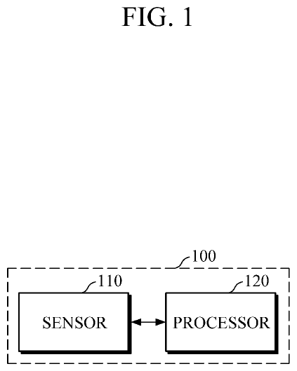

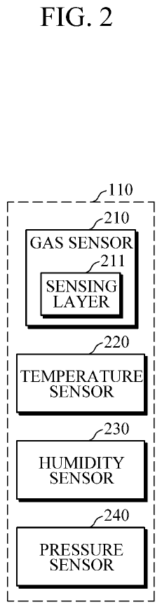

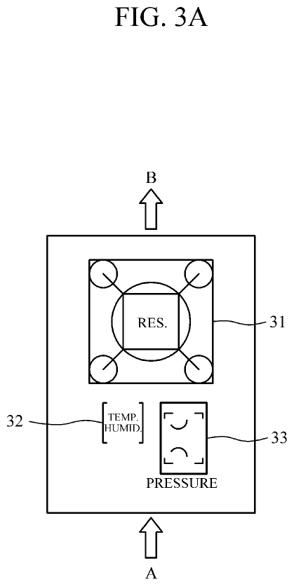

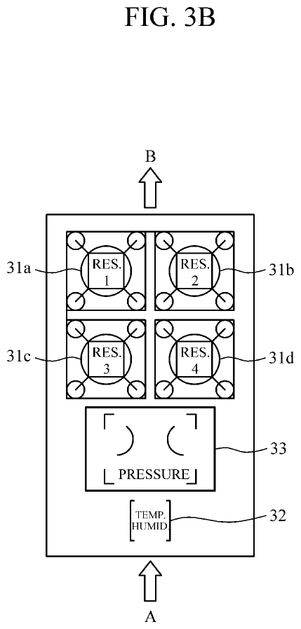

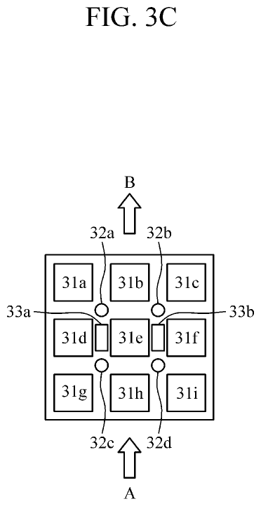

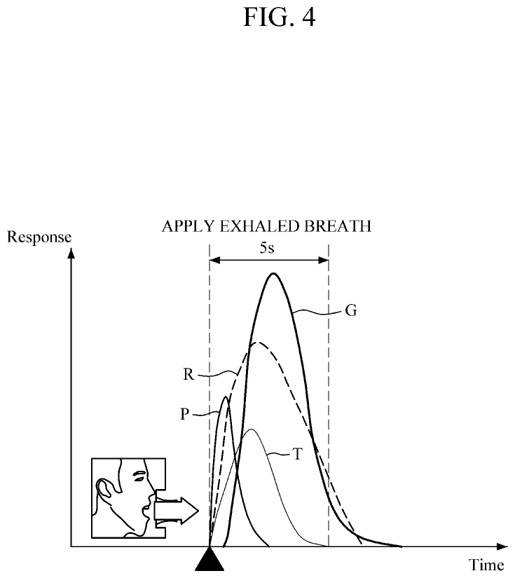

FIG. 1 is a block diagram illustrating an apparatus for analyzing an exhaled breath according to an example embodiment. FIG. 2 is a block diagram illustrating a configuration of a sensor according to an example embodiment. FIGS. 3A to 3C are diagrams illustrating examples of a structure of a sensor according to example embodiments. FIG. 4 is a diagram illustrating an example of signals related to an exhaled breath.

Referring to FIG. 1, an apparatus 100 for analyzing an exhaled breath includes a sensor 110 and a processor 120. The apparatus 100 for analyzing an exhaled breath according to example embodiments of the disclosure may be manufactured in a manner that allows a user's exhaled breath to be directly applied to the sensor 110 without separate processing.

Based on the user's exhaled breath applied to the sensor 110, the sensor 110 measures one or more signals related to the exhaled breath, including a signal indicating a target gas contained in the exhaled breath. In this case, the target gas may include sulfur compounds such as hydrogen sulfide (H₂S), Methyl mercaptan, and dimethyl sulfide. Herein, a signal related to the exhaled breath and measured by the sensor 110 may be referred to as an exhaled breath signal.

Referring to FIG. 2, the sensor 110 includes a gas sensor 210 for measuring a signal of a target gas contained in the user's exhaled breath. The gas sensor 210 may be a metal oxide semiconductor (MOS) based gas sensor 210. The metal oxide semiconductor (MOS) may be an n-type or p-type metal oxide semiconductor. However, the gas sensor 210 is not limited thereto, and may be an electrochemical sensor, a resonant sensor, an optical sensor, and the like without specific limitation.

For example, the gas sensor 210 has a sensing layer 211, whose electric resistance changes by oxidation reaction or reduction reaction with target gas molecules. The sensing layer 111 may include a gas reactive material which is oxidized or reduced by gas molecules. The gas reactive material may include metal oxide semiconductor (MOS), graphene, graphene oxide, carbon nano tube (CNT), conductive polymer, or a compound thereof, but is not limited thereto. Further, the gas reactive material may be arranged in a 2D-sheet structure. In addition, the gas reactive material may include a nanostructure e.g., nanofiber, nanotube, nanoparticle, nanosphere, nanobelt, and the like.

Furthermore, the sensing layer 111 may include a metal catalyst that is determined in consideration of gas sensitivity and selectivity. For example, the metal catalyst may be a metal nanoparticle catalyst which is uniformly bound to a gas reactive material by using apoferritin protein as a template. For example, the gas reactive material may be formed based on a metal oxide semiconductor nanofiber which is functionalized by uniformly binding alkali metal and a metal nanoparticle catalyst by electric radiation and high-temperature heat treatment.

Further, the gas sensor 210 may include a signal electrode and a heater electrode, which are coated with the sensing layer 211. The signal electrode may detect a change in an electric resistance of the sensing layer 211, and the heater electrode may adjust temperature of the sensing layer 211 to control activity of the sensing layer 211. In this case, the signal electrode and the heater electrode may be processed using microelectromechanical systems (MEMS) fabrication techniques.

The sensor 110 may further include a sensor for measuring signals related to an individual user's exhalation environment, in addition to target gas concentrations in the user's exhaled breath. For example, when the user's exhaled breath is applied in a surrounding environment, the sensor 110 may include a temperature sensor 220 for measuring temperature and/or a humidity sensor 230 for measuring humidity of the exhaled breath. In an example embodiment, the temperature sensor 220 and the humidity sensor 230 may be integrally formed with each other, but the disclosure is not limited thereto. Further, once the user's exhaled breath is applied, the sensor 110 may include a pressure sensor 240 for measuring pressure of the applied exhaled breath.

FIGS. 3A to 3C are diagrams illustrating examples of a structure of the sensor 110 according to example embodiments of the disclosure.

Referring to FIG. 3A, the sensor 110 may include a single gas sensor 31 having one sensing layer. Further, a temperature/humidity sensor 32 and a pressure sensor 33 may be disposed in a direction A-B (that is, a direction from A to B), in which the user's exhaled breath is applied. As illustrated in FIG. 3A, the temperature/humidity sensor 32 and the pressure sensor 33 may be arranged side by side in front of the single gas sensor 31. However, the arrangement is not limited thereto, and in another example, the temperature/humidity sensor 32 and the pressure sensor 33 may be disposed on both sides of the single gas sensor 31, so that each of the sensors 31, 32, and 33 may be arranged in a direction perpendicular to the direction A-B, in which the user's exhaled breath is applied.

Referring to FIGS. 3B and 3C, the sensor 110 may include a plurality of gas sensors, a plurality of temperature/humidity sensors, and a plurality of pressure sensors. However, the number and arrangement of each of the sensors are not limited to the examples to be described below.

Referring to FIG. 3B, the sensor 110 includes four gas sensors 31a, 31b, 31c, and 31d which are arranged in a 2×2 array. Each of the gas sensors 31a, 31b, 31c, and 31d may have a sensing layer, and some or all of the sensing layers may be coated with different gas reactive materials to react with different target gases, or may be formed to have different gas measuring methods. As illustrated in FIG. 3B, the gas sensors 31a, 31b, 31c, and 31d, the temperature/humidity sensor 32, and the pressure sensor 33 in the sensor 110 may be arranged in a line in the direction A-B, in which the exhaled breath is applied.

Referring to FIG. 3C, the sensor 110 includes nine gas sensor 31a, 31b, 31c, 31d, 31e, 31f, 31g, 31h, and 31i which are arranged in a 3×3 array. Each of the gas sensors 31a, 31b, 31c, 31d, 31e, 31f, 31g, 31h, and 31i may have a sensing layer, and some or all of the sensing layers may be coated with different gas reactive materials to react with different target gases, or may be formed to have different gas measuring methods. In order to accurately measure ambient temperature/humidity and pressure around each gas sensor, the sensor 110 may include a plurality of temperature/humidity sensors 32a, 32b, 32c, and 32d and a plurality of pressure sensors 33a and 33b, which may be disposed in each space between the gas sensors as illustrated in FIG. 3C.

Referring back to FIG. 1, the processor 120 may be electrically connected to the sensor 110. The processor 120 may control the sensor 110, and may obtain target gas concentrations based on the exhaled breath signal received from the sensor 110.

FIG. 4 is a diagram illustrating signals, e.g., a target gas signal G, a temperature signal T, a humidity signal R, and a pressure signal P, which are measured by the sensor 110 when an exhaled breath is applied for five seconds. The target gas, applied by a person's exhaled breath, generally acts in an area in a range of several cm², and a duration of target gas molecules is within 10 seconds, with a sampling frequency of one second or less. Further, the target gas concentration ranges from several to dozens of ppb. The target gas concentration in a person's exhaled breath is relatively greatly affected by temperature/humidity, pressure, and/or other gas molecules. By contrast, gas molecules in the air are generally transported by convection, diffusion, or wind, and may last for a relatively long period of time, e.g., several minutes or longer. In case of measuring the target gas in the air, the effect of temperature/humidity in a surrounding environment or other gas molecules, other than the effect of the target gas, is relatively minimal compared to a change caused by the target gas, and the target gas concentration ranges from several to dozens of ppm. As described above, the environment of measuring the target gas concentration in a person's exhaled breath is different from the environment of measuring the target gas concentration in the air.

In order to measure the target gas concentration in a user's exhaled breath by considering a user's individual exhalation environment, the processor 120 may obtain signal feature values of the exhaled breath signal, received from the sensor 110, and signal shape information over time of the exhaled breath signal.

For example, based on the target gas signal G, the temperature signal T, the humidity signal R, or the pressure signal P, the processor 120 may obtain, as signal feature values, initial values G₀, T₀, R₀, and P₀ obtained immediately before the exhaled breath is applied, maximum values Gₘₐₓ, Tₘₐₓ, Rₘₐₓ, and Pₘₐₓ, minimum values Gₘᵢₙ, Tₘᵢₙ, Rₘᵢₙ, and Pₘᵢₙ, difference values ΔG=Gₘₐₓ−Gₘᵢₙ, ΔT=Tₘₐₓ−Tₘᵢₙ, ΔR=Rₘₐₓ−Rₘᵢₙ, ΔP=Pₘₐₓ−Pₘᵢₙ between the maximum values and the minimum values, ratios G₀/Gₘₐₓ, T₀/Tₘₐₓ, T₀/Tₘₐₓ, P₀/Pₘₐₓ between the initial values and the maximum values, ratios G₀/Gₘᵢₙ, T₀/Tₘᵢₙ, R₀/Rₘᵢₙ, P₀/Pₘᵢₙ between the initial values and the minimum values, and ratios G₀/ΔG, T₀/ΔT, R₀/ΔR, P₀/ΔP between the initial values and the difference values. However, the feature values are not limited thereto.

Based on waveforms of the target gas signal G, the temperature signal T, the humidity signal R, or the pressure signal P, the processor 120 may obtain, as signal shape information, areas Garea, Tarea, Rarea, and Parea of a section between a first reference point and a point after a lapse of a first unit time from the first reference point, average slopes G_avg_slope, T_avg_slope, R_avg_slope, and P_avg_slope between a second reference point and a point after a lapse of a second unit time from the second reference point, instant slopes G_instant_slope, T_instant_slope, R_instant_slope, and P_instant_slope at a third reference point. However, the shape information is not limited thereto. The area of the section may be obtained by using an area under curve, integration, multiple integration, and the like. Further, the slope may be obtained by using differentiation, multiple differentiation, and the like.

In addition, by moving the first reference point, the second reference point, and the third reference point in units of a predetermined period of time (e.g., 1 second) from an initial time when the exhaled breath is applied to a time when applying of the exhaled breath is terminated, the processor 120 may obtain a plurality of section areas, a plurality of average slopes, and a plurality of instant slopes. In this case, the first reference point, the second reference point, and the third reference point may be equally set to the initial time when the exhaled breath is applied, but are not limited thereto, and may be set to different times. The first unit time and the second unit may be equally set to, for example, one second, but are not limited thereto, and may be set to different unit times.

By using the signal feature values and the shape information, the processor 120 may obtain a target gas concentration. The processor 120 may measure the target gas concentration based on the signal feature values and the shape information by applying a pre-defined target gas concentration estimation equation. The target gas concentration estimation equation may be a multiple regression equation, which is derived by using a feature value and a reference value of the target gas concentration, which are generated by using at least one or a combination of two or more of the signal feature values and the shape information obtained from the exhaled breath signal. For example, the following Equation 1 is an example of an estimation equation defined as a multiple regression equation, but the estimation equation is not limited thereto.

GC=f(G_{feature}, Gₛₕₐₚₑ)+f(R_{feature}, Rₛₕₐₚₑ)+f(T_{feature}, Tₛₕₐₚₑ)+f(P_{feature}, Pₛₕₐₚₑ) [Equation 1]

Herein, GC denotes an estimated target gas concentration, G_{feature} denotes any one or a combination of two or more of the signal feature values of the target gas; R_{feature} denotes any one or a combination of two or more of the signal feature values of humidity; T_{feature} denotes any one or a combination of two or more of the signal feature values of temperature; and P_{feature} denotes any one or a combination of two or more of the signal feature values of pressure. Further, Gₛₕₐₚₑ denotes any one or a combination of two or more of the shape information items of the target gas; Rₛₕₐₚₑ denotes any one or a combination of two or more of the shape information items of humidity; Tₛₕₐₚₑ denotes any one or a combination of two or more of the shape information items of temperature; and Pₛₕₐₚₑ denotes any one or a combination of two or more of the shape information items of pressure.

Furthermore, based on the temperature, the humidity, and/or the pressure of the exhaled breath, which are received from the temperature sensor 220, the humidity sensor 230, and/or the pressure sensor 240, respectively, the processor 120 may determine whether the exhaled breath is applied, and/or a time when applying of the exhaled breath is terminated, and the like. If at least one or a combination of two or more of the temperature, the humidity, and the pressure of the exhaled breath satisfies a defined condition, the processor 120 may determine that exhaled breath is applied by a user.

For example, when the exhaled breath is applied, the temperature, the humidity, and/or the pressure of the exhaled breath generally show an increasing trend in the temperature, the humidity, and/or the pressure. Accordingly, if any one of the temperature, the humidity, and the pressure of the exhaled breath exceeds a first threshold value or is maintained at a value greater than or equal to a second threshold value for a predetermined period of time (e.g., about five seconds) after exceeding the first threshold value, the processor 120 may determine that the exhaled breath is applied. In this case, the first threshold value and the second threshold value may be defined differently for each of the temperature, the humidity, and the pressure of the exhaled breath, and may be a fixed value which may be generally applied for a plurality of users or a value personalized to a specific user. The processor 120 may adjust the personalized value by performing calibration at predetermined intervals or in response to a user's request. In another example, if two or more of the temperature, the humidity, and the pressure of the exhaled breath satisfy a condition, which is defined for each of the temperature, the humidity, and the pressure, the processor 120 may determine that the exhaled breath is applied.

Upon determining that the exhaled breath is applied, the processor 120 may perform an algorithm for estimating a target gas concentration, and may obtain a target gas concentration based on the exhaled breath signal which is obtained until an end point of the exhaled breath. Based on the obtained target gas concentration and/or a target gas concentration analysis history, the processor 120 may analyze a user's bad breath status over time, a type of the detected target gas, a change in the bad breath status and the type of the target gas, and the like, and may monitor a health condition based on the analysis. In addition, the processor 120 may display an interface related to the target gas concentration for a user, and may provide the user with information such as the obtained target gas concentration, and recommendation, health condition, and the like based on the obtained target gas concentration.

Furthermore, based on the exhaled breath pressure received from the pressure sensor 240, the processor 120 may determine whether the exhaled breath is normally applied, an initial time of an exhaled breath signal, and/or a position at which the exhaled breath is applied (e.g., a position of the apparatus for analyzing an exhaled breath to which the exhaled breath applied), and the like. For example, upon determining that the exhaled breath is applied based on the temperature, the humidity, and/or the pressure of the exhaled breath, the processor 120 may determine a time point, at which the exhaled breath pressure exceeds a third threshold value, to be an initial time of the exhaled breath signal for analyzing a target gas concentration. In addition, if the exhaled breath pressure is not maintained at a value greater than or equal to a fourth threshold value for a predetermined period of time, the processor 120 may determine that the exhaled breath is not normally applied. Moreover, even when determining that the exhaled breath is applied, if a time point, at which the exhaled breath pressure exceeds the third threshold value, does not appear, the processor 120 may determine that a position of application of the exhaled breath is not normal. In this case, the third threshold value and the fourth threshold value may be defined for each user based on, for example, a user's exhaled breath analysis history, and may be adjusted by calibration.

Upon determining that the exhaled breath is not normally applied, the processor 120 may guide a user to normally apply the exhaled breath. For example, the processor 120 may visually display a position at which to apply the exhaled breath, or may output a graph which visually shows comparison of the pressure by the exhaled breath, which is actually applied by the user, with a reference pressure of the exhaled breath which is desired to be applied by the user, on an interface, so that the user may maintain the exhaled breath pressure at a value greater than or equal to a predetermined threshold value for a predetermined period of time, but the processor 120 is not limited thereto.

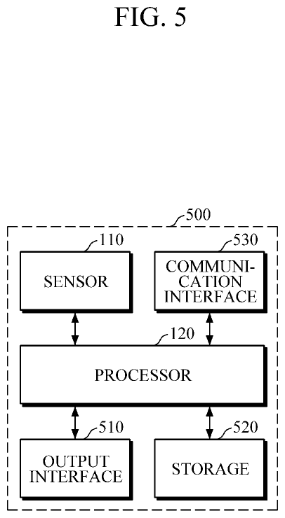

FIG. 5 is a block diagram illustrating an apparatus for analyzing an exhaled breath according to another example embodiment.

Referring to FIG. 5, an apparatus 500 for analyzing an exhaled breath includes the sensor 110, the processor 120, an output interface 510, a storage 520, and a communication interface 530. The sensor 110 and the processor 120 are described above in detail, such that a description thereof will be omitted.

The output interface 510 may output signals obtained by the sensor 110 and/or processing results of the processor 120. The output interface 510 may provide information for a user by various visual/non-visual methods using a display module, a speaker, a haptic device, and the like which are mounted in the apparatus for analyzing an exhaled breath. For example, once a target gas concentration is obtained from a user's exhaled breath, the output interface 510 may output the obtained target gas concentration. In this case, along with the target gas concentration, the output interface 510 may display detailed information, such as an exhaled breath signal including temperature, the humidity, and the pressure of the exhaled breath, a target gas, and the like, which are measured by the sensor 110, and signal feature values of the obtained exhaled breath signal, shape information, and the like, which are obtained by the processor 120. Further, the output interface 510 may display information, such as a target gas concentration history, recommendation, health condition, and the like, based on the obtained target gas concentration.

The storage 520 may store reference information required for estimating a target gas concentration, or the processing results of the sensor 110 and/or the processor 120. In this case, the reference information includes user information, such as a user's age, sex, occupation, current health condition, and the like, a target gas concentration estimation equation, and the like, but is not limited thereto.

For example, the storage 520 may include at least one storage medium of a flash memory type memory, a hard disk type memory, a multimedia card micro type memory, a card type memory (e.g., a secure digital (SD) memory, an extreme digital (XD) memory, etc.), a random access memory (RAM), a static random access memory (SRAM), a read only memory (ROM), an electrically erasable programmable read only memory (EEPROM), a programmable read only memory (PROM), a magnetic memory, a magnetic disk, and an optical disk, and the like, but is not limited thereto

The communication interface 530 may access a communication network using communication techniques to be connected to the external device. Upon connection with the external device, the communication interface 530 may receive data related to estimating a target gas concentration from the external device, and may transmit the results of the sensor 110 and/or the processor 120 to the external device. In this case, examples of the external device may include a smartphone, a tablet personal computer (PC), a desktop computer, a laptop computer, and the like, but the external device is not limited thereto.

In this case, examples of the communication techniques may include Bluetooth communication, Bluetooth low energy (BLE) communication, near field communication (NFC), wireless local access network (WLAN) communication, Zigbee communication, infrared data association (IrDA) communication, wireless fidelity (Wi-Fi) direct (WFD) communication, ultra-wideband (UWB) communication, Ant+ communication, Wi-Fi communication, and mobile communication. However, this is merely exemplary and is not intended to be limiting.

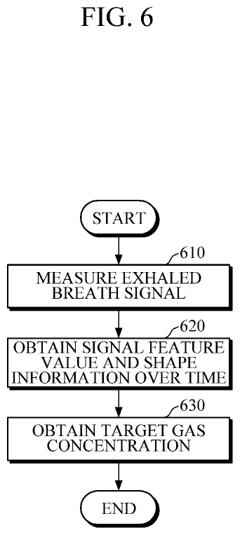

FIG. 6 is a flowchart illustrating a method of analyzing an exhaled breath according to an example embodiment.

The method of FIG. 6 is an example of a method of analyzing an exhaled breath which may be performed by any one of the apparatuses 100 and 500 for analyzing an exhaled breath according to the example embodiments of FIGS. 1 and 5.

Once exhaled breath is applied by a user, at least one of the apparatuses 100 and 500 for analyzing an exhaled breath may measure an exhaled breath signal in 610. For example, the exhaled breath signal may include a resistance value produced by reaction between the sensing layer of the gas sensor and the target gas included in the exhaled breath, the temperature, the humidity, and the pressure of the exhaled breath, and the like.

Then, at least one of the apparatuses 100 and 500 for analyzing an exhaled breath may obtain signal feature values and shape information over time based on the exhaled breath signal in 620. The signal feature values may include an initial value obtained immediately before the exhaled breath is applied, a maximum value, a minimum value, a difference value between the maximum value and the minimum value, a ratio between the initial value and the maximum value, a ratio between the initial value and the minimum value, a ratio between the initial value and the difference value, and the like. Further, the shape information may include an area of a section between a first reference point and a point after a lapse of a first unit time, an average slope between a second reference point and a point after a lapse of a second unit time, an instant slope at a third reference point, and the like. However, the information is not limited thereto.

Subsequently, at least one of the apparatuses 100 and 500 for analyzing an exhaled breath may obtain a target gas concentration based on the obtained signal feature values and the shape information in 630. For example, at least one of the apparatuses 100 and 500 for analyzing an exhaled breath may obtain the target gas concentration by using a target gas concentration estimation equation, which defines a correlation between the signal feature values, the shape information, and the target gas concentration, and is expressed in the form of a multiple regression equation. Upon obtaining the target gas concentration, at least one of the apparatuses 100 and 500 for analyzing an exhaled breath may provide the obtained target gas concentration and other information for a user.

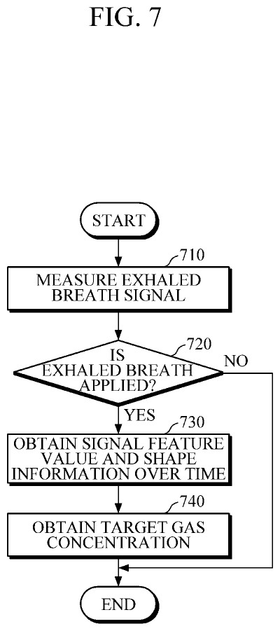

FIG. 7 is a flowchart illustrating a method of analyzing an exhaled breath according to another example embodiment.

The method of FIG. 7 is an example of a method of analyzing an exhaled breath which may be performed by any one of the apparatuses 100 and 500 for analyzing an exhaled breath according to the example embodiments of FIGS. 1 and 5.

Once an exhaled breath is applied by a user, at least one of the apparatuses 100 and 500 for analyzing an exhaled breath may measure an exhaled breath signal in 710. For example, the exhaled breath signal may include a resistance value produced by reaction between the sensing layer of the gas sensor and the target gas included in the exhaled breath, the temperature, the humidity, and the pressure of the exhaled breath, and the like.

Then, at least one of the apparatuses 100 and 500 for analyzing an exhaled breath may determine whether the exhaled breath is applied based on the measured exhaled breath signal in 720. For example, if at least one or a combination of two or more of the temperature, the humidity, and the pressure of the exhaled breath satisfies a pre-defined condition, at least one of the apparatuses 100 and 500 for analyzing an exhaled breath may determine that the exhaled breath is applied.

Subsequently, upon determining that the exhaled breath is applied in 720, at least one of the apparatuses 100 and 500 for analyzing an exhaled breath may obtain signal feature values and shape information over time from the exhaled breath signal in 730.

Next, at least one of the apparatuses 100 and 500 for analyzing an exhaled breath may obtain a target gas concentration by using the obtained signal feature values and the shape information over time in 740.

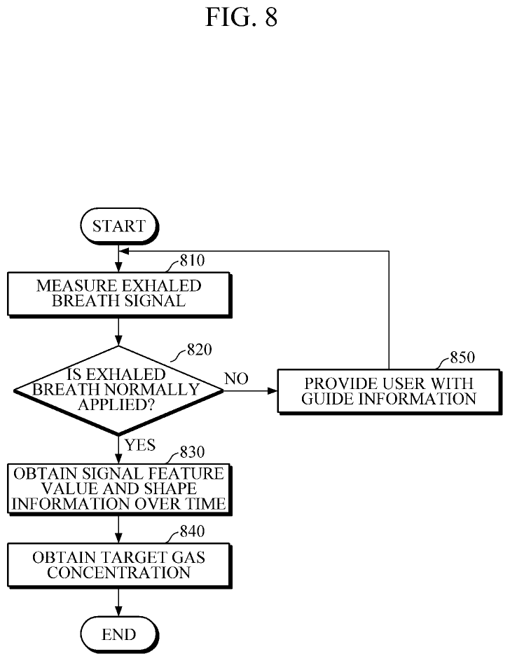

FIG. 8 is a flowchart illustrating a method of analyzing an exhaled breath according to yet another example embodiment.

The method of FIG. 8 is an example of a method of analyzing an exhaled breath which may be performed by any one of the apparatuses 100 and 500 for analyzing an exhaled breath according to the embodiments of FIGS. 1 and 5.

Once an exhaled breath is applied by a user, at least one of the apparatuses 100 and 500 for analyzing an exhaled breath may measure an exhaled breath signal in 810. For example, the exhaled breath signal may include a resistance value produced by reaction between the sensing layer of the gas sensor and the target gas included in the exhaled breath signal, the temperature, the humidity, and the pressure of the exhaled breath, and the like.

Then, at least one of the apparatuses 100 and 500 for analyzing an exhaled breath may determine whether the exhaled breath is normally applied based on the exhaled breath pressure in 820. For example, if the exhaled breath pressure is maintained at a value greater than or equal to a predetermined threshold value for a predetermined period of time, at least one of the apparatuses 100 and 500 for analyzing an exhaled breath may determine that the exhaled breath is applied in a normal manner.

Subsequently, upon determining that the exhaled breath is not normally applied in 820, at least one of the apparatuses 100 and 500 for analyzing an exhaled breath may provide a user with guide information for normally applying the exhaled breath in 850. For example, at least one of the apparatuses 100 and 500 for analyzing an exhaled breath may visually display information, including a reference exhaled breath pressure to be applied by the user, a position at which to apply the exhaled breath, and the like.

Next, upon determining that the exhaled breath is normally applied in 820, at least one of the apparatuses 100 and 500 for analyzing an exhaled breath may obtain signal feature values and shape information over time from the exhaled breath signal in 830. In this case, at least one of the apparatuses 100 and 500 for analyzing an exhaled breath may determine a time point, at which the exhaled breath pressure exceeds a predetermined threshold value, to be an initial time of receiving the exhaled breath signal for analyzing a target gas concentration, and may obtain the signal feature values and the shape information by analyzing the signal during a predetermined period of time from the initial time of receiving the exhaled breath signal.

Then, at least one of the apparatuses 100 and 500 for analyzing an exhaled breath may obtain a target gas concentration based on the obtained signal feature values and the shape information in 840.

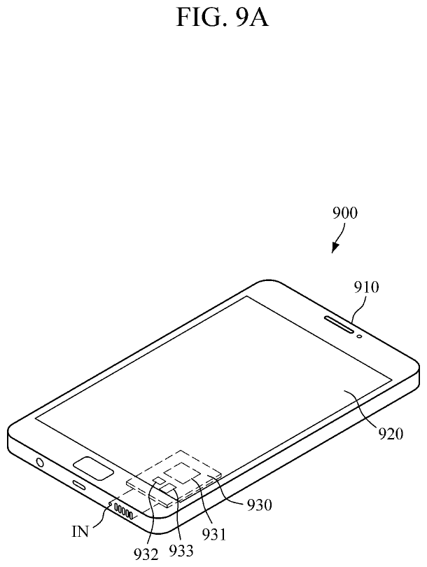

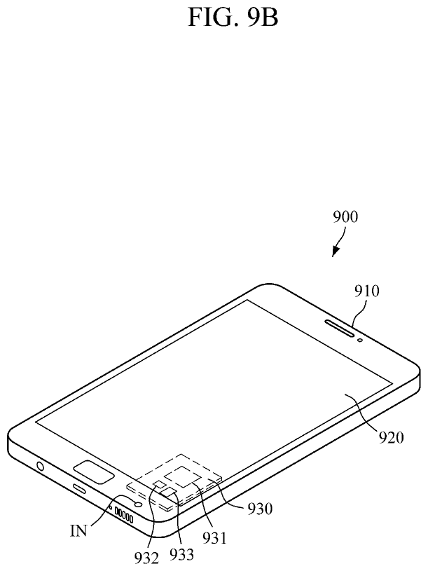

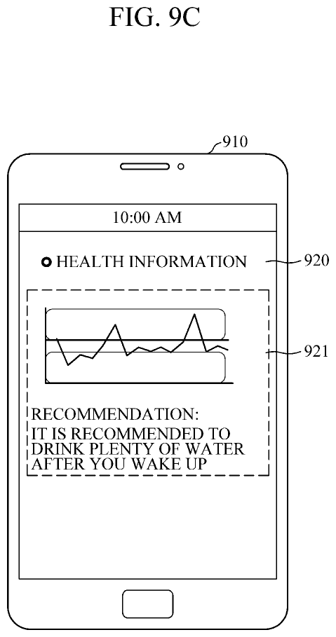

FIGS. 9A to 9C are diagrams illustrating a smart device according to an example embodiment.

FIGS. 9A to 9C illustrate a smartphone that is portable and may be used for making a call, as an example of a smart device to which an apparatus for analyzing an exhaled breath according to an example embodiment is applied. However, the smart device is not limited thereto, and may be in various forms as needed, including a tablet PC, a wearable device (e.g., a wearable device to be worn on a user's wrist), specialized medical devices used in medical institutions, and the like. The aforementioned apparatus 100 and 500 for analyzing an exhaled breath may be mounted in a smart device 900 illustrated in FIGS. 9A to 9C.

Referring to FIGS. 9A to 9C, the smart device 900 includes a main body 910 and a display 920 mounted on a front surface of the main body 910. The display 920 may display information related to analyzing a target gas concentration in the exhaled breath. The display 920 may include a touch screen for receiving a user's touch input and transmitting the received touch input to a processor.

As illustrated in FIGS. 9A and 9B, a sensor 930 may be disposed within the main body 910. As illustrated in FIGS. 9A and 9B, the sensor 930 may include a gas sensor 931, a temperature/humidity sensor 932, and a pressure sensor 933. For example, referring to FIG. 9A, the sensor 930 may be disposed within a lower portion of the main body 910, and may obtain a signal of an exhaled breath which is applied through an exhaled breath applying part IN disposed on the main body 910. In an example embodiment, the exhaled breath applying part IN may be disposed on an edge of the main body 910, as shown in FIG. 9A. In this case, the exhaled breath applying part IN, disposed on the edge of the main body 910, may also perform a function of a microphone to receive a voice input of the smart device 900. In another example, referring to FIG. 9B, the sensor 930 may be disposed within a lower portion of the main body 910, and may obtain an exhaled breath signal applied through the exhaled breath applying part IN which is separately provided at a front lower portion of the main body 910. As described above, in the case where the sensor 930 and the exhaled breath applying part IN are disposed at a lower end of the main body 910, the smart device 900 may analyze the exhaled breath of a user without being limited by time and a place even when the user speaks to the smart device 900 (e.g., speaks in a phone conversation or makes a voice command to the smart device 900). However, the arrangement of the sensor 930 and the exhaled breath applying part IN is not limited thereto.

The processor may be disposed within the main body 910, and may be electrically connected to the display 920 and the sensor 930. The processor may control the sensor 930 upon receiving a request for analyzing the exhaled breath, which may be input by touch, gesture, voice, and the like, and may output an application interface for exhaled breath analysis. Further, the processor may control the display 920 to display guide information, related to applying the exhaled breath, on the application interface for exhaled breath analysis.

Alternatively, the processor may control the sensor 930 in real time, and may monitor whether the exhaled breath is applied or whether the exhaled breath is normally applied based on an exhaled breath signal which is received from the sensor 930 in real time. Upon monitoring, if the exhaled breath is determined to be normally applied, the processor may analyze the exhaled breath by automatically performing an exhaled breath analysis algorithm.

Referring to FIG. 9C, the processor may control the display 920 to visually display information 921, such as an exhaled breath analysis result and/or recommendation based on the exhaled breath analysis result, and the like.

The example embodiments of the disclosure may be implemented as a computer-readable code written on a computer-readable recording medium. The computer-readable recording medium may be any type of recording device in which data is stored in a computer-readable manner.

Examples of the computer-readable recording medium include a ROM, a RAM, a compact disc (CD)-ROM, a magnetic tape, a floppy disc, an optical data storage, and a carrier wave (e.g., data transmission through the Internet). The computer-readable recording medium may be distributed over a plurality of computer systems connected to a network so that a computer-readable code is written thereto and executed therefrom in a decentralized manner. Functional programs, codes, and code segments needed for implementing the example embodiments may be easily deduced by programmers of ordinary skill in the art.

It should be understood that example embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. An apparatus for analyzing an exhaled breath, the apparatus comprising:
a sensor configured to obtain at least one signal related to the exhaled breath, the at least one signal including a first signal indicating a target gas contained in the exhaled breath; and
a processor configured to obtain a concentration of the target gas based on at least one signal feature value of the at least one signal and shape information over time of the at least one signal.

2. The apparatus of claim 1, wherein the at least one signal feature value comprises at least one of an initial value of a feature obtained by the sensor immediately before the exhaled breath is applied, a maximum value of the feature obtained by the sensor from the exhaled breath, a minimum value of the feature obtained by the sensor from the exhaled breath, a difference value between the maximum value and the minimum value, a ratio between the initial value and the maximum value, a ratio between the initial value and the minimum value, and a ratio between the initial value and the difference value.

3. The apparatus of claim 1, wherein the shape information comprises at least one of an area of a section in a waveform of the at least one signal between a first reference point and a point after a lapse of a first unit time from the first reference point, an average slope between a second reference point and a point after a lapse of a second unit time from the second reference point, and an instant slope at a third reference point.

4. The apparatus of claim 3, wherein the processor is further configured to obtain a plurality of section areas, a plurality of average slopes, and a plurality of instant slopes, by moving the first reference point, the second reference point, and the third reference point in units of a predetermined period of time from an initial time of obtaining the at least one signal.

5. The apparatus of claim 1, wherein the processor is further configured to obtain the concentration of the target gas by applying a pre-defined target gas estimation equation to the at least one signal feature value and the shape information.

6. The apparatus of claim 1, wherein the sensor comprises a gas sensor, the gas sensor including a sensing layer, and
wherein an electric resistance of the sensing layer changes by an oxidation reaction or reduction reaction between the sensing layer and a target gas molecule.

7. The apparatus of claim 6, wherein the sensing layer includes at least one of a metal oxide semiconductor (MOS), a graphene, a graphene oxide, a carbon nano tube (CNT), a conductive polymer, and a compound thereof.

8. The apparatus of claim 6, wherein the sensing layer comprises a metal catalyst.

9. The apparatus of claim 6, wherein the sensing layer comprises a nanostructure, the nanostructure including at least one of a nanofiber, a nanotube, a nanoparticle, a nanosphere, and a nanobelt.

10. The apparatus of claim 9, wherein the nanofiber comprises a metal oxide semiconductor nanofiber, based on uniform binding of an alkali metal and a metal nanoparticle catalyst.

11. The apparatus of claim 6, wherein the gas sensor comprises a signal electrode and a heater electrode, which are coated with the sensing layer.

12. The apparatus of claim 1, wherein the sensor comprises at least one of a temperature sensor configured to obtain a temperature of the exhaled breath, a humidity sensor configured to obtain a humidity of the exhaled breath, and a pressure sensor configured to obtain a pressure of the exhaled breath.

13. The apparatus of claim 12, wherein the processor is further configured to determine whether the exhaled breath is applied based on comparison of at least one of the temperature, the humidity, and the pressure of the exhaled breath with a predetermined threshold value.

14. The apparatus of claim 13, wherein the sensor comprises the pressure sensor, and the processor is further configured to, based on the pressure of the exhaled breath obtained by the pressure sensor, determine at least one of whether the exhaled breath is normally applied, an initial time of obtaining the at least one signal, and a position at which the exhaled breath is applied.

15. The apparatus of claim 14, wherein the processor is further configured to, based on upon a determination that the exhaled breath is not normally applied, provide information for guiding a user to re-apply the exhaled breath.

16. A method of analyzing an exhaled breath, the method comprising:
sensing, by a sensor, at least one signal related to the exhaled breath, the at least one signal including a first signal indicating a target gas contained in the exhaled breath;
obtaining at least one signal feature value of the at least one signal and shape information over time of the at least one signal; and
obtaining a concentration of the target gas based on the at least one signal feature value and the shape information.

17. The method of claim 16, wherein the obtaining the concentration of the target gas comprises obtaining the concentration of the target gas by applying a pre-defined target gas estimation equation to the at least one signal feature value and the shape information.

18. The method of claim 16, further comprising determining whether the exhaled breath is applied, based on comparison of at least one of a temperature of the exhaled breath, a humidity of the exhaled breath, and a pressure of the exhaled breath with a predetermined threshold value.

19. The method of claim 16, further comprising obtaining a pressure of the exhaled breath by using a pressure sensor, and determining, based on the pressure of the exhaled breath, at least one of whether the exhaled breath is normally applied, an initial time of obtaining the at least one signal, and a position at which the exhaled breath is applied.

20. The method of claim 19, further comprising, in response to a determination that the exhaled breath is not normally applied, providing information for guiding a user to re-apply the exhaled breath.
